(19) United States
(12) Patent Application Publication
(10) Pub. No.: US 2024/0158478 A1
(43) Pub. Date: May 16, 2024

(54) SARS-CoV-2 Antibodies and Fragments, Therapeutic Uses, Diagnostic Uses, and Compositions Related Thereto

(71) Applicants: Emory University, Atlanta, GA (US); Children's Healthcare of Atlanta, Inc., Atlanta, GA (US)

(72) Inventors: Jens Wrammert, Atlanta, GA (US); Robert Kauffman, Atlanta, GA (US); Carl Davis, Atlanta, GA (US); Mehul Suthar, Atlanta, GA (US)

(21) Appl. No.: 18/282,409

(22) PCT Filed: Mar. 15, 2022
(86) PCT No.: PCT/US2022/020322

§ 371 Date: Sep. 15, 2023

Related U.S. Application Data
(60) Provisional application No. 63/161,262, filed on Mar. 15, 2021.

(51) Int. Cl. C07K 16/10; A61K 39/00; G01N 33/569; G01N 33/577; G01N 33/58; G01N 33/60

(52) U.S. Cl. CPC C07K 16/1003; G01N 33/56983; G01N 33/577; G01N 33/581; G01N 33/582; G01N 33/60; A61K 2039/505; C07K 2317/21; C07K 2317/52; C07K 2317/565

(57) ABSTRACT

This disclosure relates to SARS-CoV-2 antibodies disclosed herein and specific binding fragments thereof, therapeutic and diagnostic uses, and compositions related thereto. In certain embodiments, this disclosure relates to antibodies disclosed herein and specific binding fragments thereof wherein the antibody or fragment specifically binds to an epitope expressed on a SARS-CoV-2 particle such as the spike protein or receptor binding domain. In certain embodiments, this disclosure relates to treating or preventing a SARS-CoV-2 or related coronavirus infection comprising administering an effective amount of an antibody disclosed herein or specific binding fragments thereof to a subject in need thereof.

### CROSS-REFERENCE TO RELATED APPLICATIONS

This application claims the benefit of U.S. Provisional Application No. 63/161,262 filed Mar. 15, 2021. The entirety of this application is hereby incorporated by reference for all purposes.

### STATEMENT REGARDING FEDERALLY SPONSORED RESEARCH OR DEVELOPMENT

This invention was made with government support under A1057266 awarded by the National Institutes of Health. The government has certain rights in this invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 21003PCT ST25.txt. The text file is 24 KB, was created on Mar. 14, 2022, and is being submitted electronically via EFS-Web.

### BACKGROUND

Some common colds are due to certain coronavirus (CoV) strains associated with mild symptoms. More dangerous human strains such as severe acute respiratory syndrome associated coronavirus (SARS-CoV-1) and SARS-CoV-2 (also referred to as COVID-19) are believed to result from coronavirus strains jumping to humans by secondary zoonotic transfers, e.g., from bats to cats and cats to humans. In humans, SARS-CoV-2 can be transferred from individuals who have mild symptoms or are asymptomatic and has caused numerous deaths worldwide. Thus, there is a need to identify treatments and preventative measures.

Liu et al. report convalescent plasma is potentially effective against a SARS-CoV-2 infection; however, not universally effective. Nat Med (2020).

Walls et al. report that the SARS-CoV-2 spike protein is involved in viral cell entry by recognizing human angiotensin converting enzyme 2 (ACE2). Cell, 2020, 180, 1-12.

Baric et al. report methods and compositions for coronavirus diagnostics and therapeutics. WO2015057666A1.

References cited herein are not an admission of prior art.

### SUMMARY

This disclosure relates to SARS-CoV-2 antibodies disclosed herein and specific binding fragments thereof, therapeutic and diagnostic uses, and compositions related thereto. In certain embodiments, this disclosure relates to antibodies disclosed herein and specific binding fragments thereof wherein the antibody or fragment specifically binds to an epitope expressed on a SARS-CoV-2 particle such as the spike protein or receptor binding domain. In certain embodiments, this disclosure relates to treating or preventing a SARS-CoV-2 or related coronavirus infection comprising administering an effective amount of an antibody disclosed herein or specific binding fragments thereof to a subject in need thereof.

In certain embodiments, this disclosure relates to chimeric antibodies disclosed herein and specific binding fragments thereof comprising one or more of the six complementarity determining regions (CDRs) of an antibody selected from antibody 4017-1B2, 4017-1D4, 4017-1H10, and 0002-2C4. In certain embodiments, the CDRs comprise the three light chain CDRs of an antibody selected from 4017-1B2, 4017-1D4, 4017-1H10, and 0002-2C4, and/or the CDRs comprise the three heavy chain CDRs of an antibody selected from 4017-1B2, 4017-1D4, 4017-1H10, and 0002-2C4, and wherein the antibody or antigen binding fragment thereof specifically binds to an epitope expressed on a SARS-CoV-2 particle.

In certain embodiments, the antibody, specific/antigen binding fragment, the light chain, or the heavy chain comprises a non-naturally occurring chimeric amino acid sequence such that there is at least one mutation is not present in naturally occurring antibodies comprising the six CDRs. In certain embodiments, the mutation is not inside/within the six CDRs. In certain embodiments, the heavy chain comprises a sequence in a constant region that is different from any sequences present in naturally derived antibodies for which the light chain variable region comprises the three light chain CDRs and the heavy chain variable region comprises the three heavy chain CDRs.

In certain embodiments, the epitope expressed on a SARS-CoV-2 particle is arrayed on a surface, expressed on the surface of a cell, or expressed at an endogenous or transfected concentration, and the antibody or specific binding fragment is bound to the epitope.

In certain embodiments, this disclosure relates to nucleic acids encoding an antibody disclosed herein or a specific binding fragment thereof, vector, or expression system, composed therein. In certain embodiments, the nucleic acid encoding an antibody or fragment is in operable combination a heterologous promoter.

In certain embodiments, this disclosure relates to pharmaceutical compositions comprising the antibody disclosed herein or specific binding fragment thereof, and a pharmaceutically acceptable carrier or excipient.

In certain embodiments, this disclosure relates methods of preventing or treating a coronavirus infection, e.g., SARS-CoV-2 infection, comprising administering an effective amount of an antibody disclosed herein or specific binding fragment thereof a subject in need thereof.

In certain embodiments, an antibody disclosed herein or specific binding fragment thereof is conjugated to a label. In certain embodiments, the label is a fluorescent tag, enzyme, or radioactive isotope.

In certain embodiments, this disclosure relates to solid surfaces conjugated to or coated with an antibody disclosed herein or specific binding fragment thereof. In certain embodiments, the solid surface is selected from a particle, magnetic particle, slide, and well.

In certain embodiments, this disclosure relates to methods of detecting a coronavirus particle or antigen, e.g., SARS-CoV-2 particle or antigen, in a sample comprising contacting an antibody disclosed herein or specific binding fragment thereof with a sample comprising a SARS-CoV-2 particle or antigen wherein the antibody disclosed herein or specific binding fragment thereof specifically binds to the SARS-CoV-2 particle or antigen; and detecting binding of the antibody disclosed herein or specific binding fragment thereof to the SARS-CoV-2 particle or antigen in a sample and thereby detecting a SARS-CoV-2 particle or antigen in a sample.

### BRIEF DESCRIPTION OF THE SEVERAL VIEWS OF THE DRAWINGS

FIG. 1 shows data from a binding assay (ELISA) to test human monoclonals binding potency against full length SARS-CoV-2 spike protein (top), and SARS-CoV-2 receptor binding domain (RBD) (bottom).

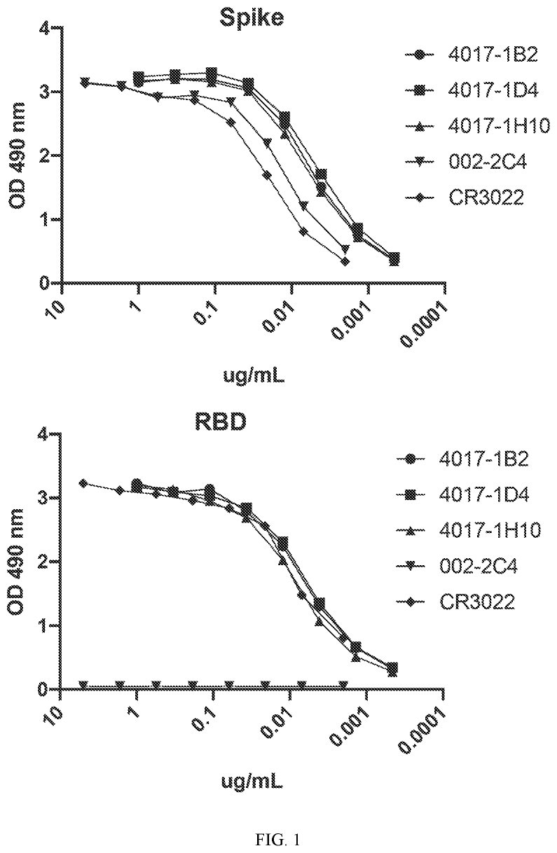

FIG. 2 shows data on SARS-CoV-2 neutralizing potency for human monoclonal antibodies 4017-1B2, 4017-1D4, and 4017-1H10. CR3022 is a negative control antibody.

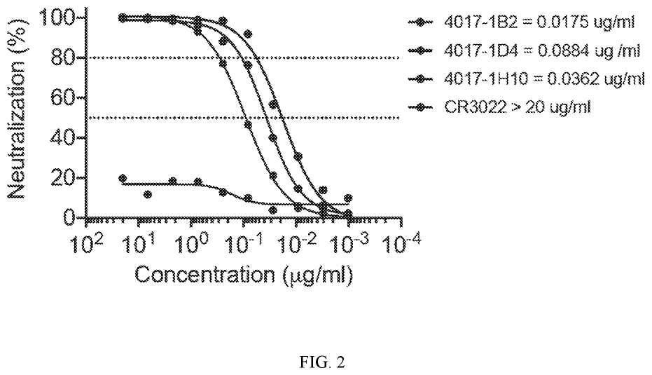

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

As used herein, the term “conjugated” refers to linking molecular entities through covalent bonds, or by other specific binding interactions, such as due to hydrogen bonding or other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

The term “specific binding agent” refers to a molecule, such as a proteinaceous molecule, that binds a target molecule with a greater affinity than other random molecules or proteins. Examples of specific binding agents include, but are not limited to, antibodies that bind an epitope of an antigen or receptors which binds a ligand. In certain embodiments, “specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

In certain embodiments, this disclosure relates to antibodies disclosed herein and specific binding fragments thereof. Experiments disclosed herein indicate antibodies disclosed herein specifically bind an epitope on full length SARS-CoV-2 spike protein, e.g., the SARS-CoV-2 receptor binding domain (RBD). Severe acute respiratory syndrome coronavirus 2 surface glycoprotein is reported to have NCBI Reference Sequence: QHD43416.1. The spike receptor binding domain is reported to be amino acids 330-583, NITNLCPFGE VFNATRFASV YAWNRKRISN 361 CVADYSVLYN SASFSTFKCY GVSPTKLNDL CFTNVYADSF VIRGDEVRQI APGQTGKIAD 421 YNYKLPDDFT GCVIAWNSNN LDSKVGGNYN YLYRLFRKSN LKPFERDIST EIYQAGSTPC 481 NGVEGFNCYF PLQSYGFQPT NGVGYQPYRV VVLSFELLHA PATVCGPKKS TNLVKNKCVN 541 FNFNGLTGTG VLTESNKKFL PFQQFGRDIA DTTDAVRDPQ TLE (SEQ ID NO: 42). Andersen et al. report six receptor binding domain (RBD) amino acids L455, F486, Q493, S494, N501 and Y505 are involved in binding to ACE2 receptors in SARS-CoV-2. Nat Med, 2020. In certain embodiments, this disclosure contemplates that an antibody epitope includes or binds to L455, F486, Q493, S494, N501 or Y505.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, the disclosure relates to antibodies disclosed herein or specific binding fragment thereof such as single chain antibodies comprising sequences disclosed herein or variants or fusions thereof wherein the amino terminal end or the carbon terminal end of the amino acid sequence are optionally attached to a heterologous amino acid sequence, label, or reporter molecule.

In certain contexts, an “antibody” refers to a protein based molecule that is naturally produced by animals in response to the presence of a protein or other molecule or that is not recognized by the animal's immune system to be a “self” molecule, i.e. recognized by the animal to be a foreign molecule and an antigen to the antibody. The immune system of the animal will create an antibody to specifically bind the antigen, and thereby targeting the antigen for elimination or degradation. A “neutralizing” antibody refers to an antibody or fragment which specifically binds to a virus particle and inhibits or reduces viral entry to target cells, e.g., reduction in viral infectivity by the binding of antibodies to the surface of viral particles (virions), thereby blocking a step in the viral replication cycle that precedes virally encoded transcription or synthesis.

It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, human antibodies, humanized antibodies, non-naturally produced synthetic antibodies, bispecific antibodies, multi-specific antibodies, chimeric antibodies, and specific binding fragments, such as single chain binding fragments (single chain antibodies). In particular, such antibodies include immunoglobulin molecules of any type (e.g., IgG, IgE, IgM, IgD, IgA and IgY), class (e.g., IgG1, IgG2, IgG3, IgG4, IgA1 and IgA2) or subclass. These antibodies may have chemical modifications.

From a structural standpoint, an antibody is typically a combination of proteins: two heavy chain proteins and two light chain proteins. The heavy chains are longer than the light chains. The two heavy chains typically have the same amino acid sequence. Similarly, the two light chains have the same amino acid sequence. Each of the heavy and light chains contain a variable segment that contains amino acid sequences which participate in binding to the antigen. The variable segments of the heavy chain typically do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

“Complementarity Determining Regions” or “CDRs” are typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain. See Kabat et al., Sequences of Proteins of Immunological Interest, 5th Ed. Public Health Service, National Institutes of Health, Bethesda, Md. (1991). CDRs may also be those residues residues 26-32 (L1), 50-52 (L2) and 91-96 (L3) in the light chain variable domain and 26-32 (H1), 53-55 (H2) and 96-101 (H3) in the heavy chain variable domain. See Chothia and Lesk, 1987, J. Mol. Biol. 196:901-917. “Framework Region” or “FR” residues are those variable domain residues other than the hypervariable region residues as herein defined.

The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc. Monoclonal antibodies directed against an antigen can be obtained from immunized mice using conventional hybridoma technology (see, e.g., U.S. Pat. No. 5,916,771). Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397.

Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, Proc. Natl. Acad. Sci. USA 91:969), and chain shuffling (U.S. Pat. No. 5,565,332).

A “chimeric antibody” is a molecule in which different portions of the antibody are derived from different immunoglobulin molecules such that the entire molecule is not naturally occurring. Examples of chimeric antibodies include those having a variable region derived from a non-human antibody and a human immunoglobulin constant region. The term is also intended to include antibodies having a variable region derived from one human antibody grafted to an immunoglobulin constant region of a predetermined sequences or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. See Jefferis et al. mAb, 1 (2009), pp. 332-338.

Smith et al. report a protocol for the production of antigen-specific human chimeric antibodies wherein antibody-secreting cells (ASCs) are isolated from whole blood collected after vaccination and sorted by flow cytometry into single cell plates. Nat Protoc. 2009; 4(3):372-84. The antibody genes of the ASCs are then amplified by RT-PCR and nested PCR, cloned into expression vectors and transfected into a human cell line. Meijer et al. report methods for isolation of human antibody repertoires with preservation of the natural heavy and light chain pairing. J Mol Biol. 2006 May 5;358(3):764-72. Wrammert et al. report using immunoglobulin variable regions isolated from sorted single ASCs to produce human monoclonal antibodies (mAbs). Nature. 2008 May 29; 453(7195): 667-671.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen.

The terms “specific binding fragment,” “antigen binding fragment,” (or simply “fragment”) and the like, of an antibody or immunoglobulin chain (heavy or light chain), as used herein, comprises a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antibodies or antigen-binding fragments thereof, for specific binding to a given epitope. In one aspect, such a fragment will retain at least one CDR present in the full-length light or heavy chain, and in some embodiments will comprise a single heavy chain and/or light chain or portion thereof. These biologically active fragments may be produced by recombinant DNA techniques, or may be produced, e.g., by enzymatic or chemical cleavage of intact antibodies. Immunologically functional immunoglobulin fragments include, but are not limited to, Fab, Fab′, F(ab′)2, Fv, domain antibodies and single-chain antibodies, and may be derived from any mammalian source, including but not limited to human, mouse, rat, camelid or rabbit. It is contemplated further that a functional portion of the antibodies disclosed herein, for example, one or more CDRs, could be covalently bound to a second protein or to a small molecule to create a therapeutic agent directed to a particular target in the body, possessing bifunctional therapeutic properties, or having a prolonged serum half-life.

A “Fab fragment” is comprised of one light chain and the CH1 domain and variable regions of one heavy chain. The heavy chain of a Fab molecule cannot form a disulfide bond with another heavy chain molecule.

An “Fc” region contains two heavy chain fragments comprising the CH1 and CH2 domains of an antibody. The two heavy chain fragments are held together by two or more disulfide bonds and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)2 molecule.

A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody may target the same or different antigens.

A “bivalent antigen binding protein” or “bivalent antibody” comprises two antigen binding sites. In some instances, the two binding sites have the same antigen specificities. Bivalent antibodies may be bispecific, see, infra.

A “multispecific antigen binding protein” or “multispecific antibody” is one that targets more than one antigen or epitope.

A “bispecific,” “dual-specific” or “bifunctional” antigen binding protein or antibody is a hybrid antigen binding protein or antibody, respectively, having two different antigen binding sites. Bispecific antibodies are a species of multispecific antigen binding protein or multispecific antibody and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553. The two binding sites of a bispecific antigen binding protein or antibody will bind to two different epitopes, which may reside on the same or different protein targets.

The term “sample”, as used herein, includes, but is not limited to, any quantity of a substance from a living thing or formerly living thing. Such living things include, but are not limited to, humans, mice, monkeys, rats, rabbits, and other animals. Such substances include, but are not limited to, blood, serum, plasma, urine, cells, organs, tissues, bone, bone marrow, lymph nodes, and skin.

The present disclosure contemplates nucleic acid molecules (DNA or RNA) that encode any such antibodies disclosed herein and specific binding fragments, as well as vector molecules (such as plasmids) that are capable of transmitting or of replication such nucleic acid molecules and expressing such antibodies, fragments, or fusion proteins fragments. The nucleic acids can be single-stranded, double-stranded, may contain both single-stranded and double-stranded portions.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. A non-limiting example of a DNA-based expression vector is pCDNA3.1, which can include includes a mammalian expression enhancer and promoter (such as a CMV promoter). Non-limiting examples of viral vectors include adeno-associated virus (AAV) vectors as well as Poxvirus vector (e.g., Vaccinia, MVA, avian Pox, or Adenovirus).

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are contemplated. For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences. A heterologous promoter refers to a promoter that originating from a different genetic source than a naturally occurring nucleic acid encoding the same protein.

Protein “expression systems” refer to in vivo and in vitro (cell free) systems. Systems for recombinant protein expression typically utilize differentiated somatic cells transfected with a DNA expression vector that contains the template. The cells are cultured under conditions such that they translate the desired protein. Expressed proteins are extracted for subsequent purification. In vivo protein expression systems using prokaryotic and somatic eukaryotic cells are well known. Also, some proteins are recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety. Mammalian cell lines available as hosts for expression are well known in the art and include, but are not limited to, immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines.

Host cells may be co-transfected with expression vectors, which may contain different selectable markers but, with the exception of the heavy and light chain coding sequences, are preferably identical. This procedure provides for equal expression of heavy and light chain polypeptides. Alternatively, a single vector may be used which encodes both heavy and light chain polypeptides. The coding sequences for the heavy and light chains may comprise cDNA or genomic DNA or both. The choice of expression vector is dependent upon the choice of host cell and may be selected so as to have the desired expression and regulatory characteristics in the selected host cell.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-(3-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: ampr, camr, tetr, blasticidinr, neor, hygr, abxr, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (at1D), UDP-glucose:galactose-1-phosphate uridyltransferasel (galT), feedback-insensitive a subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

### SARS-CoV-2 Antibodies and Specific Binding Fragments Thereof

In certain embodiments, this disclosure relates to antibodies disclosed herein and specific binding fragments derived therefrom. In certain embodiments, the antibodies are human chimeric antibodies or specific binding fragments comprising six complementarity determining regions (CDRs) of an antibody selected from antibody 4017-1B2, 4017-1D4, 4017-1H10, and 0002-2C4, wherein the CDRs comprise the three light chain CDRs of an antibody selected from 4017-1B2, 4017-1D4, 4017-1H10, and 0002-2C4, wherein the CDRs comprise the three heavy chain CDRs of an antibody selected from 4017-1B2, 4017-1D4, 4017-1H10, and 0002-2C4, and wherein the antibody or specific binding fragment thereof binds to an epitope expressed on a SARS-CoV-2 particle such as a spike protein or the receptor binding domain.

### Antibody 4017-1B2

The 4017-1B2 antibody heavy chain is encoded by the nucleic acid sequence of

(SEQ ID NO: 2)
CAGGTGCAGCTGCTGGAGTCTGGGGGAGGCGTGGTCCAGCCTGGGAGGT
CCCTGAGACTCTCCTGTGCAGCGTCTGGATTCACCTTCAGTAGTTATGG
CATGTACTGGGTCCGCCAGGCTCCAGGCAAGGGGCTGGAGTGGGTGGCA
GTTATCTGGTATGATGGAAGTAATAAATACTATGCAGACTCCGTGAAGG
GCCGATTCACCATCTCCAGAGACAATTCCAAGAACACGTTGTATCTGCA
AATGAACAGCCTGAGAGCCGAGGACACGGCTGTGTATTACTGTGCGAGA
GACGGTTTGAGCGGTTCGGGGAGTTATTATAGCCCCTTTGACTACTGGG
GCCAGGGAACCCTGGTCACCGTCTCTTCAG.

The 4017-1B2 antibody heavy chain has the amino acid sequence of

(SEQ ID NO: 10)
QVQLLESGGGVVQPGRSLRLSCAASGFTFSSYGMYWVRQAPGKGLEWVA
VIWYDGSNKYYADSVKGRFTISRDNSKNTLYLQMNSLRAEDTAVYYCAR
DGLSGSGSYYSPFDYWGQGTLVTVSS.
The heavy chain CDR1 is
(SEQ ID NO: 18)
GFTFSSYG.
The heavy chain CDR2 is
(SEQ ID NO: 19)
IWYDGSNK.
The heavy chain CDR3 is
(SEQ ID NO: 20)
ARDGLSGSGSYYSPFDY.

The 4017-1B2 antibody light chain is encoded by the nucleic acid sequence of

(SEQ ID NO: 3)
TCCTATGTGCTGACTCAGCCACCCTCAGTGTCAGTGGCCCCAGGAAAGA
CGGCCAGGATTACCTGTGGGGGAAACAACATTGGAAGTAAAAGTGTGCA
CTGGTACCAGCAGAAGCCAGGCCAGGCCCCTGTGCTGGTCATCTCTTAT
GATAGCGACCGGCCCTCAGGGATCCCTGAGCGATTCTCTGGCTCCAAGT
CTGGGAACACGGCCACCCTGACCATCAGCAGGGTCGAAGCCGGGGATGA
GGCCGACTATTACTGTCAGGTGTGGGATAGTAGTAGTTATCCCGTGGTG
TTCGGCGGAGGGACCAAGCTGACCGTCCTAG.

The 4017-1B2 antibody light chain has the amino acid sequence of

(SEQ ID NO: 11)
SYVLTQPPSVSVAPGKTARITCGGNNIGSKSVHWYQQKPGQAPVLVISY
DSDRPSGIPERFSGSKSGNTATLTISRVEAGDEADYYCQVWDSSSYPVV
FGGGTKLTVL.
The light chain CDR1 is
(SEQ ID NO: 21)
NIGSKS.
The light chain CDR2 is
(SEQ ID NO: 22)
YDSD.
The light chain CDR3 is
(SEQ ID NO: 23)
QVWDSSSYPVV.

### Antibody 4017-1D4

The 4017-1D4 antibody heavy chain is encoded by the nucleic acid sequence of

(SEQ ID NO: 4)
CAGGTGCAGCTGGTGCAGTCTGGGGCTGAGGTGAAGAAGCCTGGGTCCT
CGGTGAAGGTCTCCTGCAAGGCTTCTGGAGGCACCTTCAGCAGCTATGC
TATCAGCTGGGTGCGACAGGCCCCTGGACAAGGGCTTGAGTGGATGGGA
GGGATCATCCCTATCTTTGCTACAGCAAACTACGCACAGAAGTTCCAGG
GCAGAGTCACGATTACCGCGGACGACTCCACGAGCACAGCCTACATGGA
GCTGAGCAGCCTGAGATCTGAGGACACGGCCGTGTATTACTGTGCGAGA
GTGTCCCCTCCACGAGTGTACGGTGACTACGAGATTGGGTACTTTGACT
ACTGGGGCCAGGGAACCCTGGTCACCGTCTCTTCAG.

The 4017-1D4 antibody heavy chain has the amino acid sequence of

(SEQ ID NO: 12)
QVQLVQSGAEVKKPGSSVKVSCKASGGTFSSYAISWVRQAPGQGLEWMG
GIIPIFATANYAQKFQGRVTITADDSTSTAYMELSSLRSEDTAVYYCAR
VSPPRVYGDYEIGYFDYWGQGTLVTVSS.
The heavy chain CDR1 is
(SEQ ID NO: 24)
GGTFSSYA.
The heavy chain CDR2 is
(SEQ ID NO: 25)
IIPIFATA.
The heavy chain CDR3 is
(SEQ ID NO: 26)
ARVSPPRVYGDYEIGYFDY.

The 4017-1D4 antibody light chain is encoded by the nucleic acid sequence of

(SEQ ID NO: 5)
GACATCCAGATGACCCAGTCTCCATCCTCCCTGTCTGCATCTGTAGGAG
ACAGAGTCACCATCACTTGCCGGGCAAGTCAGAGCATTAGCAGCTATTT
AAATTGGTATCAGCAGAAACCAGGGAAAGCCCCTAACCTCCTGATCTAT
GCTGCATCCAGTTTGCAAAGTGGGGTCCCATCAAGGTTCAGTGGCAGTG
GATCTGGGACAGATTTCACTCTCATCATCAGCAGTCTGCAACCTGAAGA
TTTTGCAACTTACTACTGTCAACAGAGTTACAGTGCCCCCTACACTTTT
GGCCAGGGGACCAAGCTGGAGATCAAAC

The 4017-1D4 antibody light chain has the amino acid sequence of (SEQ ID NO: 13)

DIQMTQSPSSLSASVGDRVTITCRASQSISSYLNWYQQKPGKAPNLLIY
AASSLQSGVPSRFSGSGSGTDFTLIISSLQPEDFATYYCQQSYSAPYTF
GQGTKLEIK.
The light chain CDR1 is
(SEQ ID NO: 27)
QSISSY.
The light chain CDR2 is
(SEQ ID NO: 28)
AASS.
The light chain CDR3 is
(SEQ ID NO: 29)
QQSYSAPYT.

### Antibody 4017-11110

The 4017-1H10 antibody heavy chain is encoded by the nucleic acid sequence of

(SEQ ID NO: 6)
CAGGTGCAGCTGCAGGAGTCGGGCCCAGGACTGGTGAAGCCTTCACAGA
CCCTGTCCCTCACCTGCACTGTCTCTGGTGGCGCCATCAGCAGTGGTGG
TAACTACTGGAGCTGGATCCGCCAGCACCCAGGGAAGGGCCTGGAGTGG
ATTGGGTACATCTATTACAGTGGGATCACCTACTACAACCCGTCCCTCA
AGAGTCGACTTACCATATCAGTAGACACGTCTAAGAACCAGTTCTCCCT
GAAGCTGAGCTCTGTGACTGCCGCGGACACGGCCGTGTATTACTGTGCG
AGAGAAGTCCACTCCTATGGCTTTGACTACTGGGGCCAGGGATCCCTGG
TCACCGTCTCCTCAG

The 4017-1H10 antibody heavy chain has the amino acid sequence of

(SEQ ID NO: 14)
QVQLQESGPGLVKPSQTLSLTCTVSGGAISSGGNYWSWIRQHPGKGLEW
IGYIYYSGITYYNPSLKSRLTISVDTSKNQFSLKLSSVTAADTAVYYCA
REVHSYGFDYWGQGSLVTVSS.
The heavy chain CDR1 is
(SEQ ID NO: 30)
GGAISSGGNY.
The heavy chain CDR2 is
(SEQ ID NO: 31)
IYYSGIT.
The heavy chain CDR3 is
(SEQ ID NO: 32)
AREVHSYGFDY.

The 4017-1H10 antibody light chain is encoded by the nucleic acid sequence of

(SEQ ID NO: 7)
GACATCGTGATGACCCAGTCTCCAGACTCCCTGGCTGTGTCTCTGGGCG
AGAGGGCCACCATCAACTGCAAGTCCAGCCAGAGTGTTTTATACAGTTC
CAACAATAAGAAACACTTAGCTTGGTACCAGCAGAAACCAGGACAGCCT
CCTAAGCTGCTCATTTACTGGGCATCTGCCCGGGAATCCGGGGTCCCTG
ACCGATTCAGTGGCAGCGGGTCTGGGACAGATTTCACTCTCACCATCAG
CAGCCTGCAGGCTGAAGATGTGGCAGTTTATTACTGTCAGCAATATTAT
AATACTCCTCGGACGTTCGGCCAAGGGACCAAGGTGGAAATCAAAC

The 4017-1H10 antibody light chain has the amino acid sequence of

(SEQ ID NO: 15)
DIVMTQSPDSLAVSLGERATINCKSSQSVLYSSNNKKHLAWYQQKPGQP
PKLLIYWASARESGVPDRFSGSGSGTDFTLTISSLQAEDVAVYYCQQYY
NTPRTFGQGTKVEIK.
The light chain CDR1 is
(SEQ ID NO: 33)
QSVLYSSNNKKH.
The light chain CDR2 is
(SEQ ID NO: 34)
WASA.
The light chain CDR3 is
(SEQ ID NO: 35)
QQYYNTPRT.

Antibody 0002-2C4

The 0002-2C4 antibody heavy chain is encoded by the nucleic acid sequence of

(SEQ ID NO: 8)
GAGGTGCAGCTGGTGCAGTCTGGGGCTGAGGTGAAGAAGCCTGGGGCCT
CAGTGAAGGTTTCCTGCAAGGCATCTGGATACACCTTCACCAGGAACTA
TATGCACTGGGTGCGACAGGCCCCCGGACAAGGGCTTGAGTGGATGGGA
ATAATCAAGCCTAGTGCTGGTAGCACAACTTACGCACAGAAGTTCCAGG
GCAGAGTCACCGTGACCAGGGACACGTCCACGAGCACAGTCTTCATGGA
AGTGAGCAGCCTGAGATATGAGGACACGGCCGTGTATTATTGCGTTAGA
GATGGGAGTCGTGCTCTTGATATCTGGGGCCAAGGGACAATGGTCACCG
TCTCTTCAG

The 0002-2C4 antibody heavy chain has the amino acid sequence of

(SEQ ID NO: 16)
EVQLVQSGAEVKKPGASVKVSCKASGYTFTRNYMHWVRQAPGQGLEWMG
IIKPSAGSTTYAQKFQGRVTVTRDTSTSTVFMEVSSLRYEDTAVYYCVR
DGSRALDIWGQGTMVTVSS.
The heavy chain CDR1 is
(SEQ ID NO: 36)
GYTFTRNY.
The heavy chain CDR2 is
(SEQ ID NO: 37)
IKPSAGST.
The heavy chain CDR3 is
(SEQ ID NO: 38)
VRDGSRALDI.

The 0002-2C4 antibody light chain is encoded by the nucleic acid sequence of

(SEQ ID NO: 9)
CAGTCTGTGCTGACGCAGCCGCCCTCAGTGTCTGCGGCCCCAGGACAGA
AGGTCACCATCTCCTGCTCTGGAAGCAGCTCCAACATTGGCAATAATTA
TGTATCCTGGTACCAACAGTTCCCAGGAACAGCCCCCAAACTCCTCATT
TATGACAATAATAATCGACCCTCGGGGATTCCTGACCGATTCTCTGGCT
CCAGGTCTGGCACGTCAGCCACCCTGGGCATCACCGGACTCCGGACTGA
GGACGAGGCCGATTATTACTGCGGAACATGGGATACCACCCTGAGTGTT
TGGGTGTTCGGCGGAGGGACCAAGCTGACCGTCCTAG.

The 0002-2C4 antibody light chain has the amino acid sequence of

(SEQ ID NO: 17)
QSVLTQPPSVSAAPGQKVTISCSGSSSNIGNNYVSWYQQFPGTAPKLLI
YDNNNRPSGIPDRFSGSRSGTSATLGITGLRTEDEADYYCGTWDTTLSV
WVFGGGTKLTVL.
The light chain CDR1 is
(SEQ ID NO: 39)
SSNIGNNY.
The light chain CDR2 is
(SEQ ID NO: 40)
DNNN.
The light chain CDR3 is
(SEQ ID NO: 41)
GTWDTTLSVWV.

In certain embodiments, this disclosure contemplates variants of antibodies disclosed herein and specific binding fragments, e.g., combinations of the heavy and light chains disclosed herein and comprise light and/or heavy chains that each have more than 70%, 75%, 80%, 85%, 90%, 95%, 96%, 97%, 98% or 99% identity to the amino acid sequences disclosed herein.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG (SEQ ID NO: 43) and GGGGT (SEQ ID NO: 44) have a sequence identity of 4 out of 5 or 80%. For example, the polypeptides GGGPPP (SEQ ID NO: 45) and GGGAPPP (SEQ ID NO: 46) have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic-A V I L; Charged positive: R K H; Charged negative—D E; Polar—S T N Q. The amino acid groups are also considered conserved substitutions. Conservative modifications may be made to the heavy and light chain variable regions of antibodies disclosed herein while maintaining or improving the certain desirable functional and biochemical characteristics.

In certain embodiments, this disclosure contemplates an antibody disclosed herein and specific binding fragments, comprising: (A) a heavy chain CDR1 comprising (i) an amino acid sequence selected from the group consisting of heavy chains disclosed herein, (ii) an amino acid sequence at least 90% identical to a heavy chain amino acid sequence disclosed herein, or (iii) an amino acid sequence at least 95% identical to a heavy chain amino acid sequence disclosed herein; (B) a heavy chain CDR2 comprising (i) an amino acid sequence selected from the group consisting of heavy chains disclosed herein, (ii) an amino acid sequence at least 90% identical to a heavy chain amino acid sequence disclosed herein, or (iii) an amino acid sequence at least 95% identical to a heavy chain amino acid sequence disclosed herein; and (C) a heavy chain CDR3 comprising (i) an amino acid sequence selected from the group consisting of heavy chains disclosed herein, (ii) an amino acid sequence at least 90% identical to a heavy chain amino acid sequence disclosed herein, or (iii) an amino acid sequence at least 95% identical to a heavy chain amino acid sequence disclosed herein.

In certain embodiments, this disclosure contemplates an antibody disclosed herein and specific binding fragments, comprising: (A) a light chain CDR1 comprising (i) an amino acid sequence selected from the group consisting of light chains disclosed herein, (ii) an amino acid sequence at least 90% identical to a light chain amino acid sequence disclosed herein, or (iii) an amino acid sequence at least 95% identical to a light chain amino acid sequence disclosed herein; (B) a light chain CDR2 comprising (i) an amino acid sequence selected from the group consisting of light chains disclosed herein, (ii) an amino acid sequence at least 90% identical to a light chain amino acid sequence disclosed herein, or (iii) an amino acid sequence at least 95% identical to a light chain amino acid sequence disclosed herein; and (C) a light chain CDR3 comprising (i) an amino acid sequence selected from the group consisting of light chains disclosed herein, (ii) an amino acid sequence at least 90% identical to a light chain amino acid sequence disclosed herein, or (iii) an amino acid sequence at least 95% identical to a light chain amino acid sequence disclosed herein.

In certain embodiments, this disclosure relates to an antibody disclosed herein and specific binding fragments, wherein the antibody is selected from the group consisting of a monoclonal antibody, a Fab fragment, a Fab′ fragment, a F(ab′)2 fragment, an Fv fragment, a diabody, and a single chain antibody.

In certain embodiments, this disclosure relates to an antibody disclosed herein and specific binding fragments, wherein the antibody is a monoclonal antibody selected from the group consisting of a human chimeric antibody, and a humanized antibody.

In certain embodiments, this disclosure relates to an antibody disclosed herein, wherein the antibody is an IgG1-, IgG2-, IgG3-, or IgG4-type antibody.

In certain embodiments, the antibody disclosed herein and specific binding fragments, the light chain, or the heavy chain comprises a non-naturally occurring chimeric amino acid sequence such that there is at least one mutation that is not present in naturally occurring antibodies comprising the six CDRs. In certain embodiments, the mutation(s) are or are not inside the six CDRs.

In certain embodiments, an antibody disclosed herein and specific binding fragments comprise a human constant domain from an immunoglobulin constant region (Fc) having one or more of the following mutations: G236A, S239D, A330L, 1332E, S267E, L328F, P238D, H268F, S324T, S228P, G236R, L328R, L234A, L235A, M252Y, S254T, T256E, M428L, N434S, P329G, D265A, N297A, N297G, N297Q, F243L, R292P, Y300L, V3051, P396L, S298A, E333A, K334A, L234Y, L235Q, G236W, S239M, H268D, D270E, K326D, A330M, K334E, K326W, E333S, E345R, E430G, S440Y, L235E, and N325S, wherein the Fc mutations are in reference to positions in amino acid sequence (SEQ ID NO: 1) wherein the N-terminal amino acid serine (S) is position 119. With regard to IgG Fc mutations reported herein, the sequences are in reference to following, amino acid sequence (SEQ ID NO: 1) starting at amino acid 119 (wherein the N-terminal amino acid serine (S) is position 119):

(SEQ ID NO: 1)
STKGPSVFP LAPSSKSTSG GTAALGCLVK DYFPEPVTVS WNSGALTSGV
HTFPAVLQSS 178 GLYSLSSVVT VPSSSLGTQT YICNVNHKPS NTKVDKKVEP
KSCDKTHTCP PCPAPELLGG 238 PSVFLFPPKP KDTLMISRTP EVTCVVVDVS
HEDPEVKFNW YVDGVEVHNA KTKPREEQYN 298 STYRVVSVLT VLHQDWLNGK
EYKCKVSNKA LPAPIEKTIS KAKGQPREPQ VYTLPPSRDE 358 LTKNQVSLTC
LVKGFYPSDI AVEWESNGQP ENNYKTTPPV LDSDGSFFLY SKLTVDKSRW 418
QQGNVFSCSV MHEALHNHYT QKSLSLSPG.

It is noted that in reference to SEQ ID NO: 1, it is specifically for IgG1. IgG2, IgG3, and IgG4 will have some alternative amino acids as the same positions. For example, IgG2 contains a V at position 309 instead of L at position 309 for IgG1. IgG4 contains a F at position 234 instead of L at position 234 for IgG1.

In certain embodiments, an antibody disclosed herein and specific binding fragments comprise at least one amino acid substitution in the heavy chain constant region that is not present in naturally occurring antibodies comprising the six CDRs wherein the substitution is not within the six CDRs.

In certain embodiments, heavy chain comprises a sequence in a constant region that is different from any sequences present in naturally derived antibodies for which the light chain variable region comprises the three light chain CDRs and the heavy chain variable region comprises the three heavy chain CDRs.

In certain embodiments, with regard to any of the antibodies disclosed herein and specific binding fragments, the heavy chain comprises a human constant domain from an immunoglobulin constant region (Fc) having one, two, three, four, five, six, seven, eight, nine, ten, eleven, twelve, thirteen, fourteen, fifteen or more of the following mutations G236A, S239D, A330L, 1332E, S267E, L328F, P238D, H268F, S324T, S228P, G236R, L328R, L234A, L235A, M252Y, S254T, T256E, M428L, N434S, P329G, D265A, N297A, N297G, N297Q, F243L, R292P, Y300L, V3051, P396L, S298A, E333A, K334A, L234Y, L235Q, G236W, S239M, H268D, D270E, K326D, A330M, K334E, K326W, E333S, E345R, E430G, S440Y, L235E, N325S.

In certain embodiments, this disclosure relates to antibodies reported herein wherein the constant region comprises a mutation that activates immune responses, enhance ADC by increasing FcγRIIIa binding or decreasing FcγRIIb binding, enhance ADCP by increasing FcγRIIa binding or increased FcγRIIIa binding, enhance CDC by increasing C1q binding or hexamerization, reduce effector functions by aglycosylation, reducing FcγR and C1q binding, increasing co-engagement by increasing FcγRIIb binding, increasing FcγRIIa binding, or decreasing FcγRIIIa binding, and/or increases half-life.

In certain embodiments, this disclosure relates to antibodies disclosed herein and specific binding fragments wherein the constant region comprises a mutation in the Fc domain selected from L234A and L235A, or both.

In certain embodiments, this disclosure relates to antibodies disclosed herein and specific binding fragments wherein the constant region comprises a mutation in the Fc domain selected from L234A, L235A, and P329G, or all or combinations thereof.

In certain embodiments, this disclosure relates to antibodies disclosed herein and specific binding fragments wherein the constant region comprises a mutation in the Fc domain selected from D265A and N297A, or both.

In certain embodiments, this disclosure relates to antibodies disclosed herein and specific binding fragments wherein the constant region comprises a mutation in the Fc domain selected from D265A and N297G, or both.

In certain embodiments, this disclosure relates to antibodies disclosed herein and specific binding fragments wherein the constant region comprises a mutation in the Fc domain selected from D265A and N297Q, or both.

In certain embodiments, this disclosure relates to antibodies disclosed herein and specific binding fragments wherein the constant region comprises a mutation in the Fc domain selected from M252Y, S254T, T256E, or all or combinations thereof.

In certain embodiments, this disclosure relates to antibodies disclosed herein and specific binding fragments wherein the constant region comprises a mutation in the Fc domain selected from F243L, R292P, Y300L, V305I, P396L, or combinations thereof.

In certain embodiments, this disclosure relates to antibodies disclosed herein and specific binding fragments wherein the constant region comprises a mutation in the Fc domain selected from S239D, I332E or both.

In certain embodiments, this disclosure relates to antibodies disclosed herein and specific binding fragments wherein the constant region comprises a mutation in the Fc domain selected from S239D, I332E, A330L, or all or combinations thereof.

In certain embodiments, this disclosure relates to antibodies disclosed herein and specific binding fragments wherein the constant region comprises a mutation in the Fc domain selected from S239D, I332E, G236A, A330L, or all or combinations thereof.

In certain embodiments, this disclosure relates to antibodies disclosed herein and specific binding fragments wherein the constant region comprises a mutation in the Fc domain selected from S298A, E333A, K334A, or all or combinations thereof.

In certain embodiments, this disclosure relates to antibodies disclosed herein and specific binding fragments wherein the constant region comprises a mutation in the Fc domain selected from S298A, E333A, K334A, or all or combinations thereof.

In certain embodiments, this disclosure relates to antibodies disclosed herein and specific binding fragments wherein the constant region comprises a mutation in the Fc domain selected from L234Y, L235Q, G236W, S239M, H268D, D270E, S298A, or all or combinations thereof. In certain embodiments, this disclosure relates to antibodies disclosed herein and specific binding fragments wherein the constant region comprises a mutation in the Fc domain selected from D270E, K326D, A330M, K334E, or all or combinations thereof.

In certain embodiments, this disclosure relates to antibodies disclosed herein and specific binding fragments wherein the constant region comprises a mutation in the Fc domain selected from G236A, S239D, 1332E, or all or combinations thereof.

In certain embodiments, this disclosure relates to antibodies disclosed herein and specific binding fragments wherein the constant region comprises a mutation in the Fc domain selected from K326W, E333S or both.

In certain embodiments, this disclosure relates to antibodies disclosed herein and specific binding fragments wherein the constant region comprises a mutation in the Fc domain selected from E345R, E430G, S440Y, or all or combinations thereof.

In certain embodiments, this disclosure relates to antibodies disclosed herein and specific binding fragments wherein the constant region comprises a mutation in the Fc domain selected from E345R, E430G, S440Y, or all or combinations thereof.

In certain embodiments, this disclosure relates to antibodies disclosed herein and specific binding fragments wherein the constant region comprises a mutation in the Fc domain selected from F234A, L235A or both of IgG4.

In certain embodiments, this disclosure relates to antibodies disclosed herein and specific binding fragments wherein the constant region comprises a mutation in the Fc domain selected from H268Q, V309L, A330S, P331S or all or combinations thereof of IgG2.

In certain embodiments, this disclosure relates to antibodies disclosed herein and specific binding fragments wherein the constant region comprises a mutation in the Fc domain selected from V234A, G237A, P238S, H268A, V309L, A330S, P331S, or all or combinations thereof of IgG2.

FcgRIIb has immunosuppressive function. In certain embodiments, this disclosure relates to antibodies disclosed herein and specific binding fragments wherein the constant region comprises a mutation in the Fc domain selected from S267E, L328F or both.

In certain embodiments, this disclosure relates to antibodies disclosed herein and specific binding fragments wherein the constant region comprises a mutation in the Fc domain selected from S267E, L328F, P238D, or combinations thereof.

In certain embodiments, this disclosure relates to antibodies disclosed herein and specific binding fragments wherein the constant region comprises a mutation in the Fc domain selected from N325S and L328F or both.

Antibodies interact with the complement cascade through C1q binding enabling antibodies to activate complement-dependent cytotoxicity (CDC). In certain embodiments, this disclosure relates to antibodies disclosed herein and specific binding fragments wherein the constant region comprises a mutation that effectively active complement-dependent cytotoxicity such as those selected from S267E, H268F, S324T, or all or combinations thereof.

In certain embodiments, interaction with the immune system through Fc receptors may be unnecessary or undesirable, i.e., immune-silent antibodies. In certain embodiments, this disclosure relates to antibodies disclosed herein and specific binding fragments wherein the constant region comprises a mutation that bind the antigen but do not bind to FcgRs such as those selected from S228P, G236R, L328R, L234A, L235A, or all or combinations thereof.

In certain embodiments, this disclosure relates to antibodies disclosed herein and specific binding fragments wherein the constant region comprises a mutation in the Fc domain selected from M428L, N434S or both.

In certain embodiments, is may be desirable to have antibodies disclosed herein and specific binding fragments wherein constant region of the Fc has been to increase or decrease antibody half-life. In certain embodiments, this disclosure relates to antibodies disclosed herein and specific binding fragments wherein the constant region comprises a mutation that increases or decreases the antibodies half-life such as those selected from M252Y, S254T, T256E, M428L, N434S or all or combinations thereof.

In certain embodiments, this disclosure contemplates that a heavy chain contains at least one mutation wherein both heavy chains are not identical. In certain embodiments, this disclosure contemplates that one heavy chain may have alternative mutations than the opposite heavy chain, i.e., one of the two heavy chains contain a mutation that the other sequence does not, or one of the two heavy chains contain one or more mutations and the other heavy chain contains different mutations.

In certain embodiments, this disclosure relates to antibodies disclosed herein and specific binding fragments wherein one constant region comprises a mutation in the Fc domain selected from L234Y, L235Q, G236W, S239M, H268D, D270E, S298A, or all or combinations thereof and the opposite constant region comprises a mutation in the Fc domain selected from D270E, K326D, A330M, K334E, or all or combinations thereof.

In certain embodiments, this disclosure relates to antibodies disclosed herein and specific binding fragments wherein one constant region is IgG2. In certain embodiments, this disclosure relates to antibodies reported wherein one constant region is IgG1 and the opposite constant region is IgG2. In certain embodiments, this disclosure relates to antibodies disclosed herein and specific binding fragments wherein one constant region is IgG1 and the opposite constant region is IgG3. In certain embodiments, this disclosure relates to antibodies reported wherein one constant region is IgG2 and the opposite constant region is IgG4.

In certain embodiments, this disclosure relates to antibodies disclosed herein and specific binding fragments wherein one constant region comprises a mutation in the IgG1 Fc domain selected from L234A, L235A, or both and the opposite constant region comprises a mutation in the IgG4 Fc domain selected from F234A, L235A, or both.

This disclosure particularly contemplates the production and use of “derivatives” of any of the above-described antibodies disclosed herein and specific binding fragments. The term “derivative” refers to an antibody disclosed herein and specific binding fragments that immunospecifically binds to an antigen, which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, chimeric and/or humanized variants, as well as variants having altered CH1, hinge, CH2, CH3 or CH4 regions, e.g., variant Fc regions that exhibit enhanced or impaired effector or binding characteristics.

The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications may enhance antibody mediated effector function relative to the antibody lacking the carbohydrate modification. Carbohydrate modifications that lead to altered antibody mediated effector function are well known in the art.

The binding properties of any of the above antibodies disclosed herein and specific binding fragments can, if desired, be further improved by screening for variants that exhibit such desired characteristics. For example, such antibodies can be generated using various phage display methods known in the art. In phage display methods, functional antibody domains are displayed on the surface of phage particles which carry the polynucleotide sequences encoding them. In a particular embodiment, such phage can be utilized to display antigen binding domains, such as Fab and Fv or disulfide-bond stabilized Fv, expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead.

A derivative antibody or fragment may be modified by chemical modifications using techniques known to those of skill in the art, including, but not limited to, specific chemical cleavage, acetylation, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

Antibodies disclosed herein and specific binding fragments with increased in vivo half-lives can be generated by attaching to said antibodies or antibody fragments polymer molecules such as high molecular weight polyethylene glycol (PEG). PEG can be attached to said antibodies or antibody fragments with or without a multifunctional linker either through site-specific conjugation of the PEG to the N- or C-terminus of said antibodies or antibody fragments or via amino, thiol, or carboxylic acid groups on amino acids, e.g., epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

In certain embodiments, this disclosure relates to an antibody disclosed herein, wherein the antibody is a glycosylated IgG1 antibody. In certain embodiments, this disclosure relates to an antibody disclosed herein, wherein the antibody is not glycosylated, i.e., aglycosylated IgG1 antibody. In certain embodiments, this disclosure relates to an antibody disclosed herein having a glycosylation pattern that is different or altered from that found in the native species. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine can also be used. Addition of glycosylation sites to the antigen binding protein is conveniently accomplished by altering the amino acid sequence such that it contains one or more of the above-described tri-peptide sequences (for N-linked glycosylation sites). The alteration can also be made by the addition of, or substitution by, one or more serine or threonine residues to the starting sequence (for O-linked glycosylation sites). For ease, the antigen binding protein amino acid sequence can be altered through changes at the DNA level, particularly by mutating the DNA encoding the target polypeptide at preselected bases such that codons are generated that will translate into the desired amino acids.

Another means of increasing the number of carbohydrate moieties on the antigen binding protein is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) can be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Removal of carbohydrate moieties present on the starting antigen binding protein can be accomplished chemically or enzymatically. Chemical deglycosylation typically occurs by exposure to trifluoromethanesulfonic acid, or an equivalent compound. This treatment results in the cleavage of most or all sugars except the linking sugar (N-acetylglucosamine or N-acetylgalactosamine). Enzymatic cleavage of carbohydrate moieties on polypeptides can be achieved by the use of a variety of endo- and exo-glycosidases.

The antibodies disclosed herein and specific binding fragments may be attached to solid supports, which are particularly useful for immunoassays or purification of the target antigen or of other molecules that are capable of binding to target antigen that has been immobilized to the support via binding to an antibody disclosed herein and specific binding fragments. Such solid supports include, but are not limited to, glass, cellulose, polyacrylamide, nylon, polystyrene, polyvinyl chloride or polypropylene.

In certain embodiments, this disclosure contemplates that antibodies or fragments disclosed herein may be conjugated to an antiviral agent or cytotoxic agent. Examples for suitable groups are radioisotopes or radionuclides. Other suitable groups include toxins, therapeutic groups, or chemotherapeutic groups. Examples of suitable groups include membrane disrupting agents, calicheamicin, auristatins (e.g., monomethyl auristatin E), and geldanamycin. In some embodiments, the cytotoxic agent is coupled to antibody or fragment via spacer arms of various lengths to reduce potential steric hindrance.

### Therapeutic Methods

In certain embodiments, this disclosure relates to treating or preventing a coronavirus or SARS-CoV-2 infection comprising administering an effective amount of a antibody disclosed herein or specific binding fragment thereof to a subject in need thereof. In certain embodiments, the subject is at risk, exhibiting symptoms of, or diagnosed with a SARS-CoV-2 infection.

The terms “treat” and “treating” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a SARS-CoV-2 infection.

In certain embodiments, this disclosure relates to methods of preventing or treating a SARS-CoV-2 infection comprising administering an effective amount of an antibody, neutralizing antibody, or fragment thereof as reported herein to a subject in need thereof.

In certain embodiments, the antibody as disclosed herein or specific binding fragment thereof is administered to the subject in combination with another SARS-CoV-2 therapy, such as an antiviral agent, remdesivir, interferon-beta, hydroxychloroquine, famotidiner, ritonavir, lopinavir, risankizumab, lenzilumab, acalabrutinib, infliximab, dexamethasone, remestemcel-L, aviptadil, canakinumab, ruxolitinib, eculizumab, an antibody (mAb) that specifically binds human CD14, or combinations thereof. In certain embodiments, the antibody or fragment thereof as disclosed herein is administered to the subject in combination with a convalescent plasma transfusion.

### Diagnostic Methods

Diagnostic applications provided herein include use of the antibodies disclosed herein and specific binding fragments to detect SARS-CoV-2 or related coronavirus. In certain embodiments, this disclosure relates to methods of detecting the presence of a SARS-CoV-2 or related coronavirus in a sample comprising: contacting a sample with an antibody disclosed herein or specific binding fragment under conditions to form an antigen/antibody complex; and detecting formation of an antigen/antibody complex, whereby detection of formation of the antigen/antibody complex detects a SARS-CoV-2 or related coronavirus in the sample. In certain embodiments, a variety of assays can be employed for such detection. For example, various immunoassays can be used to detect virus particles, or molecule or peptide displayed thereon as antigen. Such immunoassays typically involve the measurement of antigen/antibody complex formation between an antigen and a SARS-CoV-2 specific antibody disclosed herein or fragment thereof.

A variety of immunoassay systems can be used, including but not limited to, radio-immunoassays (MA), enzyme-linked immunosorbent assays (ELISA) assays, enzyme immunoassays (EIA), “sandwich” assays, focus reduction neutralization assays (FRNA), gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, immunoprecipitation, competitive binding assays, and immunohistochemical staining.

In certain embodiments, this disclosure relates to methods utilizing immunoassays that can be either competitive or noncompetitive. In noncompetitive assays, for example, sandwich assays, a SARS-CoV-2 particle or antigen is bound between two antibodies. One of the antibodies is used as a capture agent and is bound to a solid surface. The other antibody is labeled and is used to measure or detect the resultant an antigen/antibody complex by e.g., visual or instrument means. In certain embodiments, antibodies disclosed herein or specific binding fragments may be the capture agent or the secondary labeled or unlabeled antibody or specific binding fragment.

In certain embodiments, the SARS-CoV-2 particle or antigen and unlabeled antibody or specific binding fragment complex can be detected by other proteins capable of specifically binding human immunoglobulin constant regions.

In certain embodiments, the non-competitive assays need not be sandwich assays. For instance, the coronavirus particle, e.g., SARS-CoV-2 particle, or antigen in the sample can be bound or placed directly to a solid surface. The presence of a particle or antigen in the sample can then be detected using a labeled or unlabeled antibody disclosed herein or specific binding fragment.

In certain embodiments, the methods further comprise contacting the surface comprising a complex of a particle or antigen and unlabeled antibody disclosed herein or specific binding fragment with secondary immunoglobulin specific antibodies conjugated to a label (labeled anti-IgG antibodies, labeled anti-IgM antibodies, and/or labeled anti-IgA antibodies).

In certain embodiments, the methods further comprise detecting the label indicating the presence of particle or antigen. In certain embodiments, the labeled secondary antibodies (e.g., labeled with a fluorescent agent, radiolabel, or an enzyme) binds the antibodies disclosed herein or specific binding fragment exposed to the solid surface. If the labeled secondary antibodies are conjugated to a fluorescent dye or radiolabel (labeled with a radio isotope), the complex formed is proportional to the degree of fluorescence when viewed with a fluorescent microscope or device for detecting radioactivity. If the labeled secondary antibody contains an enzyme, one can add a substrate that produces a signal, e.g., a color change, in the presence of the enzyme producing a signal proportional to the amount of enzyme-substrate reaction.

In certain embodiments, for any of the methods disclosed between any of the steps disclosed herein a washing step may be implemented to purify and/or separate the products from starting materials, reagents or byproducts.

In some embodiments, an antibody disclosed herein or specific binding fragments can be conjugated or otherwise linked or connected (e.g., covalently or noncovalently) to a solid support (e.g., bead, plate, slide, dish, membrane, or well). A variety of organic and inorganic polymers, both natural and synthetic can be used as the material for a solid surface. Nonlimiting examples of polymers include polyethylene, polypropylene, poly(4-methylbutene), polystyrene, polymethacrylate, poly(ethylene terephthalate), rayon, nylon, polyvinyl butyrate), polyvinylidene difluoride (PVDF), silicones, polyformaldehyde, cellulose, cellulose acetate, nitrocellulose, and the like. Other materials that can be used include, but are not limited to, paper, glass, ceramic, metal, metalloids, semi conductive materials, cements, and the like. In addition, substances that form gels, such as proteins (e.g., gelatins), lipopolysaccharides, silicates, agarose and polyacrylamides can be used. Polymers that form several aqueous phases, such as dextran, polyalkylene glycol or surfactants, such as phospholipids, long chain (12-24 carbon atoms) alkyl ammonium salts and the like are also suitable. Where the solid surface is porous, various pore sizes can be employed depending upon the nature of the system.

In certain embodiments, methods disclosed can also be carried out using a variety of solid phase systems, as well as in a dry strip lateral flow system (e.g., a “dipstick” system) wherein a fluid sample is passes over the solid surface by capillary action, i.e., liquid is soaked up as spread over the absorbent substance (e.g., paper). A labeled or unlabeled antibody disclosed herein or specific binding fragments may be contacted with the absorbent substance to interact with and detect a particle or antigen contained within the absorbent substance.

In certain embodiments, this disclosure relates to methods of measuring or quantifying a particle or antigen in a sample. In certain embodiments, methods comprise obtaining a sample from a subject; contacting the sample with labeled or unlabeled antibodies disclosed herein or specific binding fragments; measuring and/or quantifying complex formation when compared to a reference value or normalized value.

In certain embodiments, this disclosure relates to methods of measuring or quantifying viral infectivity in a sample. In certain embodiments, this disclosure relates to methods of determining whether an antibodies disclosed herein or specific binding fragment is capable of reducing infectivity of a coronavirus, e.g., SARS-CoV-2, in a sample, e.g., prior to treatment, comprising obtaining a sample from a subject infected with a coronavirus infection; contacting the sample with an antibody disclosed herein or specific binding fragment thereof and cells that expresses ACE2 on the cell membrane; culturing the cells; measuring and/or quantifying a reduction in virally-infected plaque or foci compared to a reference value or normalized value. In certain embodiments, one determines that an antibody disclosed herein or specific binding fragments are capable of reducing infectivity.

Measuring or quantifying viral infectivity may be by automated counting of plaques or foci. There are various types of plaques and foci that can be identified, e.g., using enzymatic or fluorescent labels or labeling with markers or proteins. Cells may be seeded and allowed to adhere to a surface forming multiple layers or monolayers. A sample or a control amount of virus may be added to the surface for the purpose of measuring and comparing the infectivity. The infected cell may lyse and/or spread the infection to adjacent cells where the infection cycle is repeated. If the infected cells are lysed an area will create a plaque (an area of infection surrounded by uninfected cells) which can be seen with an optical microscope or visually (e.g., by pouring off the overlay medium and adding a crystal violet solution until it has colored the cytoplasm, i.e., removing the excess solution reveals an uncolored location of dead cells) creating contrast between the cells and the opening. The samples are then imaged and analyzed using a cytometer to count the area of plaques.

Alternatively, immunostaining techniques using fluorescently labeled antibodies disclosed herein or specific binding fragments thereof may be used to detect infected host cells and infectious virus particles before an actual plaque is formed. A single or thin layer of cells that express ACE2 may be infected with various dilutions of the coronavirus. The cells and coronavirus are allowed to incubate for a period of time under an overlay medium that restricts the spread of infectious virus, creating localized clusters (foci) of infected cells. Areas are subsequently contacted with fluorescently labeled antibodies disclosed herein or specific binding fragments and fluorescence microscopy may be used to count and quantify the number or size of foci.

For diagnostic applications, an antibody disclosed herein or specific binding fragment may be labeled with a detectable labeling group. Suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, beta-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antibody via spacer arms of various lengths to reduce potential steric hindrance. Various methods for labeling proteins are known in the art and may be used.

Specific labels include optical dyes, including, but not limited to, chromophores, phosphors and fluorophores, with the latter being specific in many instances. Fluorophores can be either “small molecule” fluorophores, or proteinaceous fluorophores. By “fluorescent label” is meant any molecule that can be detected via its inherent fluorescent properties. Suitable fluorescent labels include, but are not limited to, fluorescein, rhodamine, tetramethylrhodamine, eosin, erythrosine, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable proteinaceous fluorescent labels also include, but are not limited to, green fluorescent protein (GFP), including a Renilla, Ptilosarcus, or Aequorea species of GFP, enhanced green fluorescent protein, blue fluorescent protein, and enhanced yellow fluorescent protein.

In certain embodiments, this disclosure relates to methods of detecting a SARS-CoV-2 particle or antigen in a sample comprising contacting an antibody disclosed herein or specific binding fragment thereof with a sample comprising a SARS-CoV-2 particle or antigen wherein the antibody disclosed herein or specific binding fragment thereof specifically binds to the SARS-CoV-2 particle or antigen; and detecting binding of the antibody disclosed herein or specific binding fragment thereof to the SARS-CoV-2 particle or antigen in a sample.

In certain embodiments, the antibody or specific binding fragment thereof is conjugated to a label and detecting the label is thereby detecting binding of the antibody or fragment thereof to the SARS-CoV-2 particle or antigen in a sample.

In certain embodiments, the sample is purified by gel electrophoresis or chromatography.

In certain embodiments, this disclosure relates to methods of detecting a SARS-CoV-2 particle or antigen in a sample comprising: contacting a solid surface conjugated or coated with a first antibody or fragment thereof that specifically binds a first epitope of a SARS-CoV-2 particle or antigen with a sample comprising a SARS-CoV-2 particle or antigen wherein the first antibody or fragment thereof specifically binds to the SARS-CoV-2 particle or antigen; contacting the SARS-CoV-2 particle or antigen immobilized on the sold surface with a second labeled antibody or fragment thereof that specifically binds a second epitope of SARS-CoV-2 particle or antigen providing a second labeled antibody or fragment thereof bound to the SARS-CoV-2 particle or antigen; detecting the second labeled antibody or fragment thereof bound to the SARS-CoV-2 particle or antigen thereby detecting the SARS-CoV-2 particle or antigen in the sample; wherein the first antibody or fragment thereof conjugated to the solid surface is an antibody disclosed herein.

In certain embodiments, the method further comprises the step of washing the sample to separate the sample from the solid surface wherein the SARS-CoV-2 particle or antigen is immobilized on the sold surface prior to contacting the SARS-CoV-2 particle or antigen immobilized on the sold surface with a second labeled antibody or fragment thereof.

In certain embodiments, this disclosure relates to methods of detecting a SARS-CoV-2 particle or antigen in a sample comprising: contacting a solid surface conjugated an first antibody or fragment thereof that specifically binds a first epitope of a SARS-CoV-2 particle or antigen with a sample comprising a SARS-CoV-2 particle or antigen wherein the SARS-CoV-2 particle or antigen specifically binds to the first antibody or fragment thereof conjugated to the solid surface; contacting the SARS-CoV-2 particle or antigen immobilized on the sold surface with a second labeled antibody or fragment thereof that specifically binds a second epitope of SARS-CoV-2 particle or antigen providing a second labeled antibody or fragment thereof bound to the SARS-CoV-2 particle or antigen; detecting the second labeled antibody or fragment thereof bound to the SARS-CoV-2 particle or antigen thereby detecting the SARS-CoV-2 particle or antigen in the sample; wherein the second labeled antibody or fragment thereof is an antibody disclosed herein.

In certain embodiments, the method further comprises the step of washing the sample to separate the sample from the solid surface wherein the SARS-CoV-2 particle or antigen is immobilized on the sold surface prior to contacting the SARS-CoV-2 particle or antigen immobilized on the sold surface with a second labeled antibody or fragment thereof.

### Pharmaceutical Compositions

Pharmaceutical compositions that comprise a therapeutically effective amount of one or a plurality of the antibodies disclosed herein or specific binding fragments thereof and a pharmaceutically acceptable diluent, carrier, solubilizer, emulsifier, preservative, and/or adjuvant are also provided.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapol); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants.

In certain embodiments, such compositions may influence the physical state, stability, rate of in vivo release and rate of in vivo clearance of the antibodies disclosed herein or specific binding fragment thereof. In certain embodiments, the primary vehicle or carrier in a pharmaceutical composition may be either aqueous or non-aqueous in nature. For example, a suitable vehicle or carrier may be water for injection, physiological saline solution. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute. In certain embodiments, antibody compositions may be prepared for storage by mixing the selected composition having the desired degree of purity with optional formulation agents in the form of a lyophilized cake or an aqueous solution. Further, in certain embodiments, the antibody or specific binding fragment thereof may be formulated as a lyophilizate using appropriate excipients such as sucrose.

What we claim is:

1. A non-naturally occurring chimeric antibody or specific binding fragment thereof comprising six complementarity determining regions (CDRs) of an antibody selected from 4017-1B2, 4017-1D4, 4017-1H10, and 0002-2C4,
wherein the CDRs comprise the three light chain CDRs of an antibody selected from 4017-1B2, 4017-1D4, 4017-1H10, and 0002-2C4,
wherein the CDRs comprise the three heavy chain CDRs of an antibody selected from 4017-1B2, 4017-1D4, 4017-1H10, and 0002-2C4, and
wherein the antibody or specific binding fragment thereof binds to an epitope expressed on a SARS-CoV-2 particle.

2. The antibody or specific binding fragment of claim 1 wherein the antibody, antigen binding fragment, the light chain, or the heavy chain comprises a non-naturally occurring chimeric amino acid sequence such that there is at least one mutation that is not present in naturally occurring antibodies comprising the six CDRs, wherein the mutation is not inside the six CDRs.

3. The antibody or specific binding fragment of claim 1 comprising a human constant domain from an immunoglobulin constant region (Fc) having one or more of the following mutations: G236A, S239D, A330L, 1332E, S267E, L328F, P238D, H268F, S324T, S228P, G236R, L328R, L234A, L235A, M252Y, S254T, T256E, M428L, N434S, P329G, D265A, N297A, N297G, N297Q, F243L, R292P, Y300L, V305I, P396L, S298A, E333A, K334A, L234Y, L235Q, G236W, S239M, H268D, D270E, K326D, A330M, K334E, K326W, E333S, E345R, E430G, S440Y, L235E, N325S wherein the Fc mutations are in reference to positions in amino acid sequence (SEQ ID NO: 1) wherein the N-terminal amino acid serine (S) is position 119.

4. The antibody or specific binding fragment of claim 1 comprising at least one amino acid substitution in the heavy chain constant region that is not present in naturally occurring antibodies comprising the six CDRs.

5. The antibody or specific binding fragment of claim 1, wherein the heavy chain comprises a sequence in a constant region that is different from any sequences present in naturally derived antibodies for which the light chain variable region comprises the three light chain CDRs and the heavy chain variable region comprise the three heavy chain CDRs.

6. The antibody or specific binding fragment of claim 1, wherein the epitope expressed on an SARS-CoV-2 particle is arrayed on a surface, expressed on the surface of a cell, or expressed at an endogenous or transfected concentration, and the antibody or antigen binding fragment is bound to the epitope.

7. A nucleic acid encoding an antibody or specific binding fragment of claim 1, vector, or expression system, composed therein.

8. A pharmaceutical composition comprising the antibody or specific binding fragment of claim 1, and a pharmaceutically acceptable carrier or excipient.

9. A method of preventing or treating a SARS-CoV-2 infection comprising administering an effective amount of the antibody or specific binding fragment of claim 1 to a subject in need thereof.

10. The method of claim 9, wherein the subject is at risk, exhibiting symptoms of, or diagnosed with a SARS-CoV-2 infection.

11. An antibody or specific binding fragment comprising six complementarity determining regions (CDRs) of an antibody selected from 4017-1B2, 4017-1D4, 4017-1H10, and 0002-2C4,
wherein the CDRs comprise the three light chain CDRs of an antibody selected from 4017-1B2, 4017-1D4, 4017-1H10, and 0002-2C4,
wherein the CDRs comprise the three heavy chain CDRs of an antibody selected from 4017-1B2, 4017-1D4, 4017-1H10, and 0002-2C4, and
wherein the antibody or antigen binding fragment thereof binds to an epitope expressed on a SARS-CoV-2 particle.

12. The antibody or specific binding fragment of claim 11 conjugated to a label.

13. The antibody or specific binding fragment of claim 12 wherein the label is a fluorescent tag, enzyme, or radioactive isotope.

14. A solid surface conjugated to or coated with the antibody or specific binding fragment of claim 11.

15. The solid surface of claim 13 is a particle, magnetic particle, slide, or well.

16. A method of detecting a SARS-CoV-2 particle or antigen in a sample comprising
contacting an antibody or fragment thereof that specifically binds an epitope of a SARS-CoV-2 particle or antigen with a sample comprising a SARS-CoV-2 particle or antigen wherein the antibody or fragment thereof specifically binds to the SARS-CoV-2 particle or antigen; and
detecting binding of the antibody or fragment thereof to the SARS-CoV-2 particle or antigen in a sample;
wherein the antibody or fragment thereof comprises six complementarity determining regions (CDRs) of an antibody in claim 11.

17. The method of claim 16 wherein the antibody or fragment thereof is conjugated to a label and detecting the label is thereby detecting binding of the antibody or fragment thereof to the SARS-CoV-2 particle or antigen in a sample.

18. The method of claim 16 sample is purified by gel electrophoresis or chromatography.

19. A method of detecting a SARS-CoV-2 particle or antigen in a sample comprising
contacting a solid surface conjugated or coated with a first antibody or fragment thereof that specifically binds a first epitope of a SARS-CoV-2 particle or antigen with a sample comprising a SARS-CoV-2 particle or antigen wherein the first antibody or fragment thereof specifically binds to the SARS-CoV-2 particle or antigen providing a SARS-CoV-2 particle or antigen immobilized on the solid surface;
contacting the SARS-CoV-2 particle or antigen immobilized on the sold surface with a second labeled antibody or fragment thereof that specifically binds a second epitope of SARS-CoV-2 particle or antigen providing a second labeled antibody or fragment thereof bound to the SARS-CoV-2 particle or antigen;
detecting the second labeled antibody or fragment thereof bound to the SARS-CoV-2 particle or antigen thereby detecting the SARS-CoV-2 particle or antigen in the sample;
wherein the first antibody or fragment thereof conjugated to the solid surface comprises six complementarity determining regions (CDRs) of an antibody in claim 11.

20. A method of detecting a SARS-CoV-2 particle or antigen in a sample comprising
contacting a solid surface conjugated a first antibody or fragment thereof that specifically binds a first epitope of a SARS-CoV-2 particle or antigen with a sample comprising a SARS-CoV-2 particle or antigen wherein the SARS-CoV-2 particle or antigen specifically binds to the first antibody or fragment thereof conjugated to the solid surface providing a SARS-CoV-2 particle or antigen immobilized on the sold surface;
contacting the SARS-CoV-2 particle or antigen immobilized on the sold surface with a second labeled antibody or fragment thereof that specifically binds a second epitope of SARS-CoV-2 particle or antigen providing a second labeled antibody or fragment thereof bound to the SARS-CoV-2 particle or antigen;
detecting the second labeled antibody or fragment thereof bound to the SARS-CoV-2 particle or antigen thereby detecting the SARS-CoV-2 particle or antigen in the sample;
wherein the second labeled antibody or fragment thereof comprises six complementarity determining regions (CDRs) of an antibody in claim 11.


SEQUENCE LISTING (46 sequences)

SEQ ID NO: 1 - protein, 328 aa, Artificial
  Synthetic construct
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 373 bp, Artificial
  Synthetic construct
caggtgcagctgctggagtctgggggaggcgtggtccagcctgggaggtccctgagactctcctgtgcagcgtctggattcaccttcagtagttatggcatgtactgggtccgccaggctccaggcaaggggctggagtgggtggcagttatctggtatgatggaagtaataaatactatgcagactccgtgaagggccgattcaccatctccagagacaattccaagaacacgttgtatctgcaaatgaacagcctgagagccgaggacacggctgtgtattactgtgcgagagacggtttgagcggttcggggagttattatagcccctttgactactggggccagggaaccctggtcaccgtctcttcag

SEQ ID NO: 3 - DNA, 325 bp, Artificial
  Synthetic construct
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctcttatgatagcgaccggccctcagggatccctgagcgattctctggctccaagtctgggaacacggccaccctgaccatcagcagggtcgaagccggggatgaggccgactattactgtcaggtgtgggatagtagtagttatcccgtggtgttcggcggagggaccaagctgaccgtcctag

SEQ ID NO: 4 - DNA, 379 bp, Artificial
  Synthetic construct
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttgctacagcaaactacgcacagaagttccagggcagagtcacgattaccgcggacgactccacgagcacagcctacatggagctgagcagcctgagatctgaggacacggccgtgtattactgtgcgagagtgtcccctccacgagtgtacggtgactacgagattgggtactttgactactggggccagggaaccctggtcaccgtctcttcag

SEQ ID NO: 5 - DNA, 322 bp, Artificial
  Synthetic construct
gacatccagatgacccagtctccatcctccctgtctgcatctgtaggagacagagtcaccatcacttgccgggcaagtcagagcattagcagctatttaaattggtatcagcagaaaccagggaaagcccctaacctcctgatctatgctgcatccagtttgcaaagtggggtcccatcaaggttcagtggcagtggatctgggacagatttcactctcatcatcagcagtctgcaacctgaagattttgcaacttactactgtcaacagagttacagtgccccctacacttttggccaggggaccaagctggagatcaaac

SEQ ID NO: 6 - DNA, 358 bp, Artificial
  Synthetic construct
caggtgcagctgcaggagtcgggcccaggactggtgaagccttcacagaccctgtccctcacctgcactgtctctggtggcgccatcagcagtggtggtaactactggagctggatccgccagcacccagggaagggcctggagtggattgggtacatctattacagtgggatcacctactacaacccgtccctcaagagtcgacttaccatatcagtagacacgtctaagaaccagttctccctgaagctgagctctgtgactgccgcggacacggccgtgtattactgtgcgagagaagtccactcctatggctttgactactggggccagggatccctggtcaccgtctcctcag

SEQ ID NO: 7 - DNA, 340 bp, Artificial
  Synthetic construct
gacatcgtgatgacccagtctccagactccctggctgtgtctctgggcgagagggccaccatcaactgcaagtccagccagagtgttttatacagttccaacaataagaaacacttagcttggtaccagcagaaaccaggacagcctcctaagctgctcatttactgggcatctgcccgggaatccggggtccctgaccgattcagtggcagcgggtctgggacagatttcactctcaccatcagcagcctgcaggctgaagatgtggcagtttattactgtcagcaatattataatactcctcggacgttcggccaagggaccaaggtggaaatcaaac

SEQ ID NO: 8 - DNA, 352 bp, Artificial
  Synthetic construct
gaggtgcagctggtgcagtctggggctgaggtgaagaagcctggggcctcagtgaaggtttcctgcaaggcatctggatacaccttcaccaggaactatatgcactgggtgcgacaggcccccggacaagggcttgagtggatgggaataatcaagcctagtgctggtagcacaacttacgcacagaagttccagggcagagtcaccgtgaccagggacacgtccacgagcacagtcttcatggaagtgagcagcctgagatatgaggacacggccgtgtattattgcgttagagatgggagtcgtgctcttgatatctggggccaagggacaatggtcaccgtctcttcag

SEQ ID NO: 9 - DNA, 331 bp, Artificial
  Synthetic construct
cagtctgtgctgacgcagccgccctcagtgtctgcggccccaggacagaaggtcaccatctcctgctctggaagcagctccaacattggcaataattatgtatcctggtaccaacagttcccaggaacagcccccaaactcctcatttatgacaataataatcgaccctcggggattcctgaccgattctctggctccaggtctggcacgtcagccaccctgggcatcaccggactccggactgaggacgaggccgattattactgcggaacatgggataccaccctgagtgtttgggtgttcggcggagggaccaagctgaccgtcctag

SEQ ID NO: 10 - protein, 124 aa, Artificial
  Synthetic construct
QVQLLESGGGVVQPGRSLRLSCAASGFTFSSYGMYWVRQAPGKGLEWVAVIWYDGSNKYYADSVKGRFTISRDNSKNTLYLQMNSLRAEDTAVYYCARDGLSGSGSYYSPFDYWGQGTLVTVSS

SEQ ID NO: 11 - protein, 108 aa, Artificial
  Synthetic construct
SYVLTQPPSVSVAPGKTARITCGGNNIGSKSVHWYQQKPGQAPVLVISYDSDRPSGIPERFSGSKSGNTATLTISRVEAGDEADYYCQVWDSSSYPVVFGGGTKLTVL

SEQ ID NO: 12 - protein, 126 aa, Artificial
  Synthetic construct
QVQLVQSGAEVKKPGSSVKVSCKASGGTFSSYAISWVRQAPGQGLEWMGGIIPIFATANYAQKFQGRVTITADDSTSTAYMELSSLRSEDTAVYYCARVSPPRVYGDYEIGYFDYWGQGTLVTVSS

SEQ ID NO: 13 - protein, 107 aa, Artificial
  Synthetic construct
DIQMTQSPSSLSASVGDRVTITCRASQSISSYLNWYQQKPGKAPNLLIYAASSLQSGVPSRFSGSGSGTDFTLIISSLQPEDFATYYCQQSYSAPYTFGQGTKLEIK

SEQ ID NO: 14 - protein, 119 aa, Artificial
  Synthetic construct
QVQLQESGPGLVKPSQTLSLTCTVSGGAISSGGNYWSWIRQHPGKGLEWIGYIYYSGITYYNPSLKSRLTISVDTSKNQFSLKLSSVTAADTAVYYCAREVHSYGFDYWGQGSLVTVSS

SEQ ID NO: 15 - protein, 113 aa, Artificial
  Synthetic construct
DIVMTQSPDSLAVSLGERATINCKSSQSVLYSSNNKKHLAWYQQKPGQPPKLLIYWASARESGVPDRFSGSGSGTDFTLTISSLQAEDVAVYYCQQYYNTPRTFGQGTKVEIK

SEQ ID NO: 16 - protein, 117 aa, Artificial
  Synthetic construct
EVQLVQSGAEVKKPGASVKVSCKASGYTFTRNYMHWVRQAPGQGLEWMGIIKPSAGSTTYAQKFQGRVTVTRDTSTSTVFMEVSSLRYEDTAVYYCVRDGSRALDIWGQGTMVTVSS

SEQ ID NO: 17 - protein, 110 aa, Artificial
  Synthetic construct
QSVLTQPPSVSAAPGQKVTISCSGSSSNIGNNYVSWYQQFPGTAPKLLIYDNNNRPSGIPDRFSGSRSGTSATLGITGLRTEDEADYYCGTWDTTLSVWVFGGGTKLTVL

SEQ ID NO: 18 - protein, 8 aa, Artificial
  Synthetic construct
GFTFSSYG

SEQ ID NO: 19 - protein, 8 aa, Artificial
  Synthetic construct
IWYDGSNK

SEQ ID NO: 20 - protein, 17 aa, Artificial
  Synthetic construct
ARDGLSGSGSYYSPFDY

SEQ ID NO: 21 - protein, 6 aa, Artificial
  Synthetic construct
NIGSKS

SEQ ID NO: 22 - protein, 4 aa, Artificial
  Synthetic construct
YDSD

SEQ ID NO: 23 - protein, 11 aa, Artificial
  Synthetic construct
QVWDSSSYPVV

SEQ ID NO: 24 - protein, 8 aa, Artificial
  Synthetic construct
GGTFSSYA

SEQ ID NO: 25 - protein, 8 aa, Artificial
  Synthetic construct
IIPIFATA

SEQ ID NO: 26 - protein, 19 aa, Artificial
  Synthetic construct
ARVSPPRVYGDYEIGYFDY

SEQ ID NO: 27 - protein, 6 aa, Artificial
  Synthetic construct
QSISSY

SEQ ID NO: 28 - protein, 4 aa, Artificial
  Synthetic construct
AASS

SEQ ID NO: 29 - protein, 9 aa, Artificial
  Synthetic construct
QQSYSAPYT

SEQ ID NO: 30 - protein, 10 aa, Artificial
  Synthetic construct
GGAISSGGNY

SEQ ID NO: 31 - protein, 7 aa, Artificial
  Synthetic construct
IYYSGIT

SEQ ID NO: 32 - protein, 11 aa, Artificial
  Synthetic construct
AREVHSYGFDY

SEQ ID NO: 33 - protein, 12 aa, Artificial
  Synthetic construct
QSVLYSSNNKKH

SEQ ID NO: 34 - protein, 4 aa, Artificial
  Synthetic construct
WASA

SEQ ID NO: 35 - protein, 9 aa, Artificial
  Synthetic construct
QQYYNTPRT

SEQ ID NO: 36 - protein, 8 aa, Artificial
  Synthetic construct
GYTFTRNY

SEQ ID NO: 37 - protein, 8 aa, Artificial
  Synthetic construct
IKPSAGST

SEQ ID NO: 38 - protein, 10 aa, Artificial
  Synthetic construct
VRDGSRALDI

SEQ ID NO: 39 - protein, 8 aa, Artificial
  Synthetic construct
SSNIGNNY

SEQ ID NO: 40 - protein, 4 aa, Artificial
  Synthetic construct
DNNN

SEQ ID NO: 41 - protein, 11 aa, Artificial
  Synthetic construct
GTWDTTLSVWV

SEQ ID NO: 42 - protein, 253 aa, Artificial
  Synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 43 - protein, 6 aa, Artificial
  Synthetic construct
GGGGGG

SEQ ID NO: 44 - protein, 5 aa, Artificial
  Synthetic construct
GGGGT

SEQ ID NO: 45 - protein, 6 aa, Artificial
  Synthetic construct
GGGPPP

SEQ ID NO: 46 - protein, 7 aa, Artificial
  Synthetic construct
GGGAPPP